(19) United States
(12) Patent Application Publication
(10) Pub. No.: US 2022/0059231 A1
(43) Pub. Date: Feb. 24, 2022

(54) WATCHDOG SMART FACE PROTECTIVE DEVICE AND SMART FACE SHIELD FOR INFECTION CONTROL

(71) Applicant: Ahmad SALEH, Dubai (AE)

(72) Inventor: Ahmad SALEH, Dubai (AE)

(21) Appl. No.: 17/404,419

(22) Filed: Aug. 17, 2021

Related U.S. Application Data
(60) Provisional application No. 63/089,690, filed on Oct. 9, 2020.
(60) Provisional application No. 63/067,166, filed on Aug. 18, 2020.

(51) Int. Cl. G16H 50/30; G16H 50/80; G16H 10/60; G16H 50/20; G06F 1/16

(52) U.S. Cl. CPC G16H 50/30; G16H 50/80; G06F 1/163; G16H 50/20; G16H 10/60

(57) ABSTRACT

A smart face protective device (SFPD) for infection control, where the smart face protective device is configured to detect health and safety (H&S) conditions of the user and/or a person passing in proximity of the user and conduct actions based on the detection. The smart face protective device has a moveable face shield portion configured to be automatically adjusted between a secured positon and an unsecured position based on a command signal received at the device. A smart face shield that can detect the temperature of a user and the user's distance with respect to another person. The smart face shield has a shell, a pad, and a circuitry. The pad is fixed to the shell and the circuitry allows for the operation of the smart features of the smart face shield.

### CROSS-REFERENCE TO RELATED APPLICATIONS AND PRIORITY

This patent application claims priority from U.S. Provisional Patent Application No. 63/067,166 filed Aug. 18, 2020 and U.S. Provisional Patent Application No. 63/089,690 filed Oct. 9, 2020. Each of these patent applications are herein incorporated by reference in their entirety.

### FIELD OF THE INVENTION

The present invention relates to personal protective equipment, and more particularly to a watchdog smart face protective device and smart face shield for wear by a user in order to prevent or reduce the spread of infectious diseases such as COVID-19.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Hundreds of million of students from prekindergarten through primary and secondary grades globally have had their education disrupted by school closures amid the continuing spread of a new coronavirus COVID-19, a figure the U.N. agency dealing with the crisis says is unprecedented. There are school closures of some kind in dozens of countries on three continents with hundreds of millions of students around the world facing upheaval, including many countries that have shut schools nationwide. The United Nations has warned of the unparalleled scale and speed of the educational disruption being caused by coronavirus.

Schools are starting to take precautions to prevent virus outbreaks, including canceling trips, teaching students to wash their hands for an appropriate period of time with soapy water and sending reassuring messages to parents. A few schools have closed, and others are considering doing that if necessary.

Other schools have implemented the wearing of face shields or masks as mandatory precautionary measures. Face shields are simple, transparent screens that cover the face and help prevent infectious droplets from entering the eyes, nose and mouth. They are usually worn for blocking splashes and sprays from reaching the face and preventing workers from touching their faces.

Schools, hospitals and other institutions have been facing increasing challenges of controlling and managing the wear of masks and face shields within their institutions, in addition to the challenges for taking precautionary measures as well as corrective measures for enhancing the safety and health conditions of their members. Though these measures have proven to be one of the most implemented and recommended preventive measures among others, there is a lot of difficulty also associated with their usage. In fact, face shield is often preferred over face masks for several reasons. Firstly, face shields cover the whole face from the head extending all the way below the chin, instead of just the mouth and nose area (as in the case of face masks). As a result, the face shield prevents the user from touching his/her own face and eyes that may trigger spreading of the virus. It has been observed in a recently conducted study that face shields reduce the inhalation exposure of the user by 96 percent within 18 inches of a cough.

A further advantage associated with the use of face shields is that they are comfortable and convenient to be worn by the user and being made of transparent material, allows others to see the user' s expressions or read their lips, resulting in better communication without the need to take off the face shield.

The conventional face shields as described above, do have a number of advantages, but there is still room for improvement. It is therefore an object of the invention to provide a smart face protective device comprising of a face shield and a smart face shield which offer additional functionality to the users and enhance the performance of the conventional face shields.

### SUMMARY OF THE INVENTION

As an aspect of the invention, there is provided a smart face protective device and a smart face shield which is configured to be worn by a user for infection control, wherein the smart face protective device and smart face shield are configured to detect health and safety (H&S) conditions of the user and/or persons passing in proximity of the user and take actions based on the detection.

In an embodiment of the invention, these actions comprise precautionary or notification measures such as triggering a notification signal (such as an alarm signal) and/or recording the H&S detected data for subsequent access. The data can also be sent remotely to an external device.

In an embodiment of the invention, the H&S conditions relate to a temperature of the user or a person passing in proximity of the user, the social distance between the user and another person (intruding person), a motion by the user or another person to touch the user's face.

In an embodiment of the invention, the smart face protective device comprises a notification unit for providing notification signals to the user and/or other users or third parties based on the H&S detection. The device can include a light (LED for example), a vibrator, a display, a sound system or another interface to communicate the notification to the user and/or other persons.

In an embodiment of the invention, the device generates a notification signal (alarm signal) in case the user's temperature is above a predefined threshold (for example 38.5 degrees Celsius).

In an embodiment of the invention, the device generates a notification signal (alarm signal) in case the social distance between the user and another person (intruding person) is below a predefined social distance threshold (for example 6 feet).

In an embodiment of the invention, the device generates a notification signal (alarm signal) in case the user removes the smart face protective device. This last feature can be associated to a time period, such that the notification signal is triggered if the smart face protective device is removed and not put it back before the expiration of a given predefined time, for example 1 minute. For example, a blinking led can be provided and activated by the device as an indication of the device being removed. The blinking light can remain active until this is being reset by a user. This is helpful for infants at school which may provide teachers and admin staff with an indication whether an infant has removed the device during a given time period. Also, a vibration can be activated to vibrating the device until the device is worn again by the user.

In an embodiment of the invention, the device may trigger a buzz/vibration and/or activate a light on the device or play a voice recording (such as “keep social distance”) based on the H&S detection.

In an embodiment of the invention, the smart face protective device comprises a notification unit configured to notify a health/safety risk condition such as a high temperature above the predefined temperature threshold. The notification unit is configured to notify the user or other users through a user interface such as a screen, a led, a voice message, or a vibrator. In an embodiment of the invention, the notification unit is configured to be connected to a remote administration device through a data network (such as WiFi) to notify an administrator (such as a teacher or school director) of the presence of a health/safety risk associated to the user.

In an embodiment of the invention, the thermal detector is configured to monitor the body temperature of others persons passing or being in proximity of the user (also called intruding person or intruding persons) and the controller is configured to notify the user or other users or third parties when a body temperature of the intruding person is detected to be above the predefined temperature threshold (for example 37.8 or 38 degrees Celsius). The purpose of this feature is to alert the user that the high temperature of the intruding person might be indicative of the intruding person being sick or having been infected by a contagious disease (such as COVID-19), and in this case the user and/or other users can take action to prevent/reduce the risks of the intruding person infecting the user.

In an embodiment of the invention, the thermal detector monitors the body temperature of intruding persons being within a predefined social safety distance threshold which is preferably around 6 feet (however this can vary depending on the application). The reason there is a social safety distance threshold is because only a user located at a distance below the threshold poses a risk to the user, and therefore there is no need in obtaining the temperature of an intruding person that is located at a distance beyond the social safety distance threshold.

In an embodiment of the invention, the intruding persons are other users (for example other kids at the same school) having similar smart face protective devices (SFPDs), and wherein each one of the smart face protective devices comprises an inter-device communication unit in communication with the controller and with a memory. In an embodiment of the invention, the inter-device communication units associated to the various face protective devices are configured to exchange health/safety data relative to their respective users (for example the identity of the intruding persons passing within the social safety distance threshold, and the temperature of the intruding users) and to store this data inside the memory of their respective SFPDs.

In an embodiment of the invention, the data exchanged by a SFPD comprises an indication of any health/safety risk associated to its respective user (for example an indication of high temperature of the user, or a measure of the temperature itself), and wherein the SFPD generates a notification/alarm signal when/if the temperature of the intruding person as obtained by the inter-communication unit is above the predefined temperature threshold. Preferably, the identity of the intruding user (or associated face protective device) is stored inside the memory of the user's face protective device which can be retrieved at any future time for tracking and identification purposes of infected persons.

In an embodiment of the invention, the smart face protective device is designed to reduce the risk of infection by the COVID-19, and the predefined temperature threshold is set around 38 degrees Celsius. Preferably, the predefined temperature threshold is in the range of 37.5 degrees Celsius and 38.5 degrees Celsius. A temperature above this predefined temperature threshold is indicative of the presence of a fever which might be indicative of the presence of a virus such as COVID-19.

In an embodiment of the invention, the smart face protective device comprises a user interface having an input/output interface configured to enable a user to set and change the predefined temperature threshold and/or the social safety distance threshold depending on the application and requirements.

In an embodiment of the invention, the thermal detector comprises a non-contact infrared sensor configured to detect the temperature of the user. In an embodiment of the invention, the non-contract infrared sensor is also configured to detect the temperature of intruding persons passing in proximity of the user (preferably those who are within the social safety distance threshold only).

In an embodiment of the invention, the Health & Safety Monitoring Unit further comprises a social distance detector for detecting presence of an intruding person within a predefined minimum social safety distance threshold of the user, and wherein the notification unit sends a notification signal (alarm signal) when the intruding person is detected within the predefined minimum social safety distance threshold. Preferably, the inter-device communication unit obtains the identity of the intruding person (or associated device) and stores the information inside the memory.

In an embodiment of the invention, the social safety distance threshold is around 6 feet. This is the minimum distance required between two persons in order to prevent/reduce the risks of communicating droplets and body fluids between the two persons and therefore being contaminated by COVID-19 or other infections. This minimum threshold can preferably be set and changed depending on the application and requirements using the input/output interface of the face protective device.

In an embodiment of the invention, the SFPD is used to act as a barrier between the user's portal portion of the face and the external environment, and is made of a suitable material depending on the application. In an embodiment of the invention, the SFPD is made of a solid material such as transparent plastic or solidified plastic (such as acrylic) or glass acting as barrier to droplets and body fluids without obstructing the vision of the user.

In an embodiment of the invention, the SFPD comprises of an ultra-violet (UV) light configured to sanitize the SFPD. In another embodiment of the invention, the SFPD comprises an air ionizer configured to create an ionized layer of air acting as barrier to infected droplets and body fluids.

In an embodiment of the invention, the SFPD is configured to be used in an indoor environment for preventing or reducing the spread of COVID-19. The indoor environment can be any type of organization or institution or establishment such as an education institution, an elderly care center, a hospital or a shopping mall. In an embodiment of the invention, the SFPD is particularly useful for children in the age range of 2 to 15 years old.

In an embodiment of the invention, the SFPD has a unique identifier allowing to uniquely identify the SFPD. In an embodiment of the invention, the SFPD is associated to a specific user and the unique identifier enables to uniquely identify the user associated to the SFPD. In an embodiment of the invention, the unique identifier is stored inside the memory of the SFPD and accessed to/used by the controller depending on the application. The unique identifier can be static or dynamic such that it can be updated to reflect the identity of the user using the SFPD at any given time. In an embodiment of the invention, the input/output interface of the SFPD is configured to enable the set up/change the unique identifier based on the identity of the user using the device.

In an embodiment of the invention, the Health & Safety Monitoring Unit is an external Health & Safety Monitoring Unit located remote from the smart face protective device. In this case, the communication between the controller and the Health & Safety Monitoring Unit is a remote wireless communication (through a data network such as WiFi or Bluetooth or any other suitable wireless communication technology) conducted through the communication unit of the SFPD.

In an embodiment of the invention, the Health & Safety Monitoring Unit comprises an identification system configured to identify the user. The identification system can for example be an RFID system wherein the RFID reader can be located at the remote (external) Health & Safety Monitoring Unit and the RFID tag is located at the SFPD. The Health & Safety Monitoring Unit identifies the user, monitors his body temperature and sends a signal to the SFPD of the user (through the controller) in order to make a proper notification to the user or other parties. In another embodiment, the thermal detector of the Health & Safety Monitoring Unit screens the body temperature of other persons passing or being located in proximity of the user (the intruding persons), and sends a signal to the SFPD of the user (through the communication unit connected to the controller) in order to notify the user when the temperature of an intruding person is beyond the predefined temperature threshold.

In an embodiment of the invention, the Health & Safety Monitoring Unit is mechanically coupled to the SFPD.

In an embodiment of the invention, the SFPD is configured to be mechanically coupled or affixed to a functional article such as a furniture. For example, the functional article can be a cubicle, a desk, a bed, a wheelchair, an internal component of a vehicle, etc.

In an embodiment of the invention, the SFPD comprises a face shield unit comprising a face shield providing a safety/security barrier to the user from the external environment and intruding persons. In one example, the face shield is a physical shield made of a solid material (such as a plastic or glass window for example). The face shield can be mounted along a peripheral portion of the functional article (such as a student cubicle/desk or a wheelchair or a patient bed) and configured to provide a security zone to the user (school student or patient) and protect him/her from infected intruding persons (or the opposite, protecting the other persons from an infected user).

As an example, SFPD can be fixed to cubicles/desks of school kids at school such that each cubicle/desk is equipped with a corresponding SFPD for providing school kids with a safe environment for them and others while studying. Each kid student in this case can have his own safe cubicle/desk environment with the minimum intrusion possible where the SFPD is only activated when required (for example when there is another intruding kid approaching the cubicle/desk or when the kid himself has some symptoms of infection such as high temperature). As another example, the SFPD can be fixed to patients' beds in hospitals such that each patient bed is equipped with a corresponding SFPD for providing patients with a safe environment for them and others while being hospitalized. Each patient in this case can have his own safe bed environment with the minimum intrusion possible where SFPD is only activated when required (for example when there is another intruding patient or healthcare professional approaching or when the patient himself has some symptoms of infection such as high temperature). In another example, the SFPD can be fixed to wheelchairs for providing persons with special needs with a safe environment for them and others while commuting.

In an embodiment of the invention, the SFPD is a portable device configured to be carried or worn by the user. In this case, the face shield unit of the SFPD comprises a face shield preferably made of solid material and, the face shield unit further comprises a motor in connection with the controller configured to open/close the face shield based on the instructions received from the controller which are based on the signals received from the Health & Safety Monitoring Unit.

In an embodiment of the invention, the SFPD comprises a mounting structure configured to be carried or worn by the user, and a transparent face shield coupled to the mounting structure. Preferably, the face shield is removable.

In an embodiment of the invention, the smart face protective device (SFPD) comprises:
a face shield unit;
a Health & Safety Monitoring Unit for monitoring a health and safety condition of a user (or other users preferably those passing in proximity of the user) and outputting a signal with an indication of the health and safety condition of the user (H&S signal);
a notification unit for generating a notification signal based on the H&S signal received.

In an embodiment of the invention, the notification unit provides notification signals to the user and/or other users or third parties based on the H&S detection. In an embodiment of the invention, the notification unit stores data related to the H&S detection inside a memory for future access. In another embodiment, the data is transmitted (preferably wirelessly) to an external device for storage and future access.

In an embodiment of the invention, the Health & Safety Monitoring Unit comprises a thermal detector for monitoring a body temperature of the user, and wherein the notification unit generates a notification signal indicative of the high temperature when the body temperature of the user is above a predefined temperature threshold. In an embodiment of the invention, the predefined temperature threshold is around 38 degrees Celsius. For example, when the temperature of the user is above 38 degrees, this can be an indication that the user is sick and carrying the COVID-19 virus.

In an embodiment of the invention, the thermal detector is adapted to measure the temperature of another person passing in proximity of the user. When a high temperature (beyond the predefined temperature threshold) is detected, a notification signal (alarm signal) is generated by the notification unit. This is to protect the user from the risk of being infected in case the person passing in proximity of the user is infectious.

In an embodiment of the invention, the notification unit can generate a signal indicative of a normal situation (absence of danger) in case where there is a low risk of communicating an infectious disease between the user and other persons, for example when the user does not have medical symptoms of being infected by a virus (i.e. when the user's temperature is below the temperature threshold) and in absence of other persons passing in proximity of the user.

In an embodiment of the invention, the thermal detector comprises a non-contact infrared sensor. Preferably, the non-contact infrared sensor is mounted in a position pointing at a forehead of the user when the SFPD is being worn by the user, for measuring the user's temperature through the user's forehead. The non-contact infrared sensor can for example be mounted at the SFPD itself or on a mounting structure coupled/part of the SFPD such as a component which is designed to hold the SFPD in place. In an embodiment of the invention, the infrared sensor is mounted at the internal side of the SFPD such that it points at the forehead of the user when it is being worn by the user. In another embodiment, the infrared sensor line of sight projects outwardly away from the user to detect the temperature of an intruding person passing in proximity of the user (within the minimum social safety distance threshold for example).

In an embodiment of the invention, the Health & Safety Monitoring Unit comprises a social distance detector for detecting presence of another user within a predefined minimum social safety distance threshold, and wherein the notification unit generates an alarm signal when another user is detected within the predefined minimum social safety distance threshold. This is in order to reduce the risk of passing infectious droplets or fluid between the user and other persons passing in proximity of the user within the minimum social safety distance threshold. Preferably, the social safety distance threshold is around 6 feet.

In an embodiment of the invention, the social distance detector comprises a proximity detector which can comprise at least one of an infrared sensor, an ultrasound sensor, and an optical sensor, or a laser sensor.

In an embodiment of the invention, the user is a member of a community (such as a school or a hospital or a care center) and wherein other community members are wearing a similar SFPD, such that each SFPD has a unique identifier configured to uniquely identify the SFPD and the user/community member wearing the SFPD. In an embodiment of the invention, the unique identifiers associated to the intruding persons of the user (those breaching the minimum social safety distance threshold) are obtained by the SFPD of the user and stored inside the memory.

In an embodiment of the invention, the social distance detector comprises of an ultra-wide band based distance measuring unit which is configured to measure the distance between the user and other members of the community wearing a similar smart face protective device. In an embodiment of the invention, tracking information associated to a user and all intruding persons are stored within the memory of the smart face protective device of the user or an external server. This information can be referred to at any later stage to track and assess health risks of the user or other persons who have been in direct contact with the user at any given time.

In an embodiment of the invention, the Health & Safety Monitoring Unit comprises a hands-to-face detector for detecting a hand gesture of the user to touch his face, and wherein the controller is configured to generate a notification signal (alarm signal) when the hand gesture of the user to touch his face is detected. In an embodiment of the invention, the hands-to-face detector comprises a proximity sensor mounted in a position capable of detecting presence of the user's hands in proximity of the user's face. In an embodiment of the invention, the proximity sensor is an infrared sensor. However, the proximity sensor can also be an ultra-sound sensor or any other suitable type of sensors capable of detecting presence of hands in proximity of the user's face.

In an embodiment of the invention, the proximity sensor is mounted at the SFPD and positioned such that it detects presence of the user's hands when these come in proximity of the user's face (or the user's portal portion of the face).

In an embodiment of the invention, the proximity sensor is an infrared sensor mounted along the perimeter of a face opening portion of the SFPD.

In another embodiment of the invention, the hands-to-face detector comprises infrared sensors positioned within a bracelet pointing towards the user' s wrist and configured to detect a hands-to-face movement based on an image of the wrist captured by said infrared sensors.

In an embodiment of the invention, the SFPD comprises a mounting structure for receiving the face shield unit, the Health & Safety Monitoring Unit and the notification unit.

In an embodiment of the invention, the face shield unit covers all the portals of entry for this virus: the eyes, the nose, and the mouth. Moreover, the supply chain is significantly more diversified than that of face masks, so availability is much greater. Face shields are the easiest type of personal protective equipment (PPE) to make. They typically consist of just two parts: a visor that covers the face and which is usually made of plastics such as polycarbonate, propionate, acetate, polyvinyl chloride (PVC), and polyethylene terephthalate glycol (PETG); and a method of holding the visor in place, such as a headband or strap. The strap can be made of moulded plastic, 3D-printed plastic or even elastic.

As another aspect of the invention, the face shield unit comprises a moveable face shield portion configured to be automatically adjusted between a secured and unsecured positions (close and open the face shield respectively, for example) based on a command signal received by the device.

In an embodiment of the invention, the automatic adjustment is conducted without a manual force by a user, preferably through a motor connected to the face shield for automatically opening and closing the face shield based on the command signal. The command signal can originate from the user, or can be generated automatically by the device at the occurrence of a certain event such as the detection of a H&S condition associated to the user or a person passing in proximity of the user.

In an embodiment of the invention, the face shield has a face shield opening and where the moveable portion of the face shield is configured to cover the face shield opening when the device is in the secured position, and to uncover it otherwise when the device is in the unsecured position.

Preferably, in an embodiment of the invention, the command signal is a command signal from the user, such as for example a voice signal, a touch signal, or a text signal.

In an embodiment of the invention, the SFPD comprises a voice command unit for receiving a voice command from the user and converting the voice command from the user into a command signal for automatically adjusting the face shield based on the voice command received. The voice command unit preferably comprises a micro as part of the user interface of the device, and is preferably connected to the controller of the device.

In an embodiment of the invention, the command signal is a voice signal such as “open shield” and “close shield” for respectively opening and closing the moveable part of the face shield, or any other commands as suitable.

In an embodiment of the invention, the command signal received by the device is based on the health and safety conditions of the user or persons passing in proximity of the user.

In an embodiment of the invention, the automatic adjustment (opening and closing the face shield) is conducted without any human intervention based on a command signal received by the device based on the health and safety conditions of the user or persons passing in proximity of the user.

In an embodiment of the invention, the SFPD comprises a safety feature to ensure the safety operation of the opening and closing of the face shield, such as for example a motion sensor to detect a motion of the user' s hands across the face shield opening and restrict the face shield from closing when/if it is the case.

As a further aspect of the invention, there is provided a smart face protective system for preventing or reducing spread of infectious diseases within a community comprising community members, the system comprising:
Smart face protective devices (SFPDs) in accordance with the various embodiments of the present invention for use respectively by the community members such that each SFPD is uniquely identified and associated to a single community member;
A SFPD Admin System in communication with the plurality of SFPDs through a wireless data network for remotely administrating the SFPDs and for receiving and processing health and safety data related to the community members as collected by the SFPDs and for outputting protective and/or corrective healthcare measures based on the collected data.

As a further aspect of the invention, there is provided a SFPD network for preventing or reducing spread of infectious diseases within a community comprising community members, the Network comprising:
N number of SFPD Groups (90A, 90B, . . . , 90N), where each SFPD Group (90A, 90B, . . . , 90N) comprises K number of SFPDs (10A, 10B, . . . , 10K);
N number of SFPD Control Devices (80A, 80B, . . . , 80N), where each SFPD Control Device (80A, 80B, . . . , 80N) is connected/configured to be connected to one SFPD Group (90A, 90B, . . . , 90N);
a SFPD Admin System connected/configured to be connected to the N SFPD Control Devices.
   where N is greater than or equal to 2 and K is greater than or equal to 2

Preferably, the SFPDs of the network are smart face protective devices according to the various embodiments of the present invention.

As a further aspect of the invention, there is provided a method of preventing or reducing spread of infectious diseases within a community comprising community members, the method comprising:
dividing the community members into N groups, such that each group comprise K members;
providing the K community members with K smart face protective devices in accordance with the various embodiments of the invention;
providing N Admin Control Devices for respectively managing and controlling the N groups such that each Amin Control Device is paired and connected to K smart face protective devices associated to a given group for simultaneously managing and controlling said K smart face protective devices.

Preferably, the method further comprises providing a SFPD system in communication Admin Control Devices for remotely administrating the Admin Control Devices and for receiving and processing health and safety data related to the community members as collected by the smart face protective devices and for outputting protective and/or corrective healthcare measures based on the collected data.

As another aspect of the invention, there is provided a smart face shield comprising a shell, a pad configured to be fixed to the shell, and a circuitry fixed to the pad for the operation of smart features of the face shield device.

As a further aspect of the invention, there is provided a smart face shield comprising a shell, a pad configured to be fixed to the shell, wherein the pad comprises openings configured to receive a circuitry fixed to the pad for the operation of smart features of the smart face shield.

In an embodiment of the invention, the pad can be configured to house the circuitry.

In an embodiment of the invention, the shell is a U shaped sheet made of a bendable material. The bendable material can be plastic, aluminum, or stainless steel.

In an embodiment of the invention, the pad can be made of foam or other soft material. The foam can be lining foam or memory foam.

In an embodiment of the invention, the pad comprises a garment cover for covering the pad, the garment cover having a socket adapted to receive the shell for removably fixing the pad to the shell. The garment cover can be made of a washable material.

In an embodiment of the invention, the smart face shield further comprises of a shield connected to the shell.

In an embodiment of the invention, the shield is made of polyethylene terephthalate glycol (PETG) or other transparent material.

In an embodiment of the invention, the shield can be removably connected to the shell.

In an embodiment of the invention, the smart face shield further comprises of a forehead housing positioned at about the forehead of a user when the smart face shield is being worn, the forehead housing being configured to receive at a first part of the circuitry.

In an embodiment of the invention, the first part of the circuitry comprises a contactless heat sensor configured to detect the user's temperature using the user's forehead.

In an embodiment of the invention, the first part of the circuitry further comprises a proximity sensor for detecting a proximity distance with an intruding person in proximity of the user.

In an embodiment of the invention, the proximity sensor is a laser sensor configured to measure the distance with the intruding person.

In an embodiment of the invention, the pad is configured to keep the forehead housing at a predefined distance from the forehead of the user while the smart face shield is being worn such that the forehead housing and related electronic components do not touch the user's forehead.

In an embodiment of the invention, the circuitry comprises an indicator panel for providing a signal when the temperature of the user is beyond a predefined temperature threshold and when an intruding person is below a predefined social distance threshold from the user.

In an embodiment of the invention, the indicator panel may comprise a plurality of indicators each connected to at least one sensor in the circuitry.

In an embodiment of the invention, the predefined temperature threshold is around 38.5° C., and wherein the predefined social distance threshold is about 2 meters.

In an embodiment of the invention, the smart face shield further comprises of a switching button and a USB port positioned on the shell.

In an embodiment of the invention, there is provided a process of manufacturing a smart face shield comprising a shell, a pad, and a circuitry.

In an embodiment of the invention, the process comprising providing the shell in a U shaped configuration such that the shell is bendable and flexibly mountable around the head of a user, cutting the pad for receiving at least one circuit component within the pad and fixing the pad to the shell.

In an embodiment of the invention, the shell is bent and laser-cut from a bendable sheet material.

In an embodiment of the invention, the process may comprise including one or more of Arduino nano, Lithium Battery Charger (Micro USB), resistors, buzzers, tracker, VL53L0X sensor, slide switch, female to female jumper wires, and MLX90614 contactless temperature sensor as circuit components within the pad.

To further clarify the advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings.

### BRIEF DESCRIPTION OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which:

FIG. 1 is a Smart Face Protective Device (SFPD) for infection control according to an embodiment of the present invention;

FIG. 2 is a Smart Face Protective Device (SFPD) in accordance with another embodiment of the present invention;

FIG. 3 is a Health & Safety Monitoring Unit of the SFPD for monitoring health & safety conditions in accordance with an embodiment of the present invention;

FIG. 4 illustrates a SFPD Face Shield Unit in accordance with an embodiment of the present invention;

FIG. 5 illustrates a SFPD User Interface in accordance with an embodiment of the present invention;

FIG. 6 illustrates a SFPD network with a group of SFPDs in a remote data communication with a SFPD Admin System in accordance with an embodiment of the present invention;

FIG. 7 illustrates a SFPD Network with a plurality of SFPDs divided into groups controlled by respective Admin Control Devices and an SFPD Admin System in accordance with an embodiment of the present invention; and

FIG. 8 illustrates a SFPD Control Device for simultaneously controlling/managing a group of SFPDs in accordance with an embodiment of the present invention.

FIG. 9 illustrates an overall schematic view of a smart face shield, in accordance with an embodiment of the present disclosure.

FIG. 10 illustrates a back view of the smart face shield, in accordance with an embodiment of the present disclosure;

FIGS. 11 A-F illustrates different views of the smart face shield worn by a user, in accordance with an embodiment of the present disclosure;

FIGS. 12 A-D illustrates different views of the smart face shield, in accordance with an embodiment of the present disclosure;

FIGS. 13 A-C illustrates dimensions corresponding to the smart face shield, in accordance with an embodiment of the present disclosure; and

FIGS. 14A and 14 B illustrates different views of the positioning of circuit elements in the smart face shield, according to an embodiment of the present disclosure.

FIG. 15 illustrates a flow diagram depicting a process of manufacturing a smart face shield.

Further, skilled artisans will appreciate that elements in the drawings are illustrated for simplicity and may not have necessarily been drawn to scale. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein

### DETAILED DESCRIPTION OF THE INVENTION

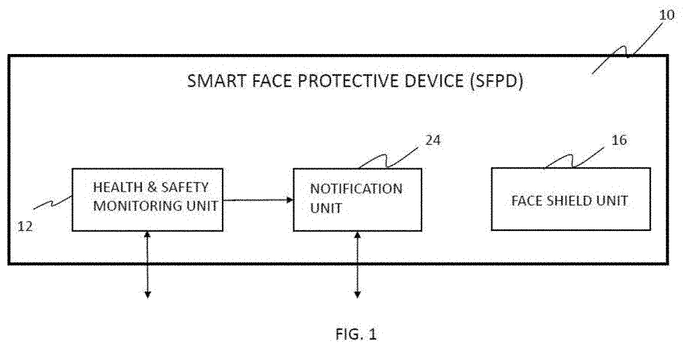

The aspects of the invention will be described in conjunction with FIGS. 1-15. In the Detailed Description, reference is made to the accompanying figures, which form a part hereof, and in which is shown by way of illustration specific embodiments in which the invention may be practiced. It is to be understood that other embodiments may be utilized and logical changes may be made without departing from the scope of the present invention. The following detailed description, therefore, is not to be taken in a limiting sense, and the scope of the present invention is defined by the appended claims.

Referring to FIG. 1, there is provided a first embodiment of a smart face protective device (SFPD) 10 for preventing or reducing spread of infectious droplets through a portal portion of a user's face. The SPFD 10 comprises a Health & Safety Monitoring Unit 12, a notification unit 24 and a face shield unit 16 for monitoring a health & safety condition related to a user and for generating a notification signal (such as an alarm signal) based on the monitored health & safety condition related to the user. The monitoring of the health and safety condition of the user and the generation of the notification signal are conducted in real time. The SFPD 10 can be a wearable device or a non-wearable device designed to be mounted to a functional article related to the user such as a wheel chair, a bed, a cubicle, a desk, a vehicle component or so on. The SFPD 10 is preferably uniquely identified and associated to the user.

The Health & Safety Monitoring Unit 12 is in electrical communication with the notification unit 24 directly (or through a controller 14 depending on the design and application) for monitoring health & safety conditions related the user (at least one condition) and, based on the monitoring, sending a notification signal (such as an electrical signal) reflecting the monitored health & safety conditions to the notification unit 24.

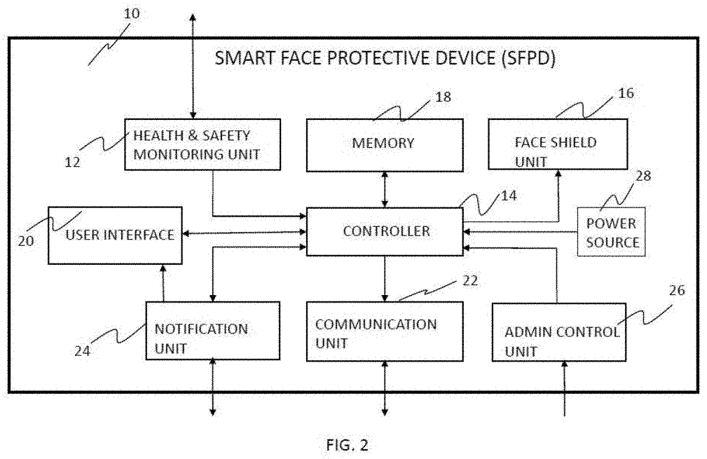

Referring to FIG. 2, in another embodiment, the smart face protective device (SFPD) 10 further comprises at least a part of the following components a controller 12, a memory 18, a user interface (also named input/output interface) 20, a communication unit (also called inter-device communication unit) 22, a notification unit 24, an admin control unit 26, and a power source 28.

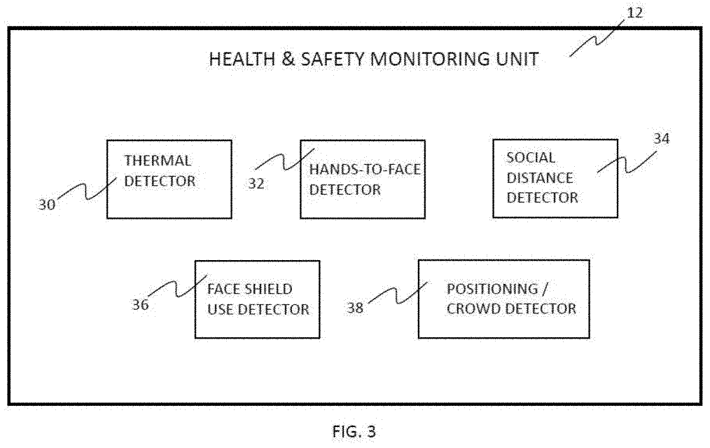

Referring to FIG. 3, the Health & Safety Monitoring Unit 12 preferably comprises a thermal detector 30 for monitoring the body temperature of the user and/or of another person. The thermal detector 30 preferably comprises a first infra-red (IR) sensor for monitoring and measuring the body temperature of the user (who is wearing/using the SFPD). The infra-red (IR) sensor can be a non-contact sensor designed to point and read the body temperature from the forehead of the user. When the SFPD 10 is configured to be a head wearable device, the IR non-contact sensor is preferably mounted to the head wearable device in such a manner that the IR non-contact sensor points to the forehead of the user or another member of the user's body from which the user's body temperature can be read. As the health & safety condition of the user also depends on the health conditions of persons coming into direct physical contact with the user, the thermal detector 30 is preferably configured to detect the body temperature of other persons coming into direct physical contact with the user (also called persons being in proximity of the user or intruding persons). In this case, the thermal sensor 30 preferably comprises a second thermal sensor (such as IR sensor) being configured to measure the temperature of persons coming into direct contact with the user (preferably those coming within the minimum social safety distance threshold).

In an embodiment of the invention, the notification unit 24 generates a notification signal (alarm signal) indicative that the body temperature of the user (or the intruding person) is above the predefined temperature threshold, when the body temperature measured is above the predetermined threshold. The detection of the body temperature by the thermal detector 30, and the generation of the notification signal indicative of the high temperature are all conducted in real time and within a very short time period (preferably less than 1-2 seconds) in order to reduce infection risks.

The Health & Safety Monitoring Unit 12 preferably further comprises a Hands-to-Face Detector 32 for detecting a hand gesture of the user (or another person such as another child trying to reach out to the user's face) to touch the user's face. The Hands-to-Face Detector 32 preferably comprises one or more sensors (is referred to as a hands-to-face sensor for purpose of language simplification) configured to detect either a particular movement, shape or location of one or both hands which is indicative of a hand gesture to reach out to the user's face. The sensor can be a proximity sensor such as an IR sensor, an ultrasound sensor or any other suitable type of sensors. The hands-to-face sensor can be an IR sensor for detecting presence of hands in proximity of the user's face. In case the SFPD 10 comprises head wearable device, the IR sensor can be mounted to the head wearable device in such a way to detect presence of hands (or another object) in proximity of the user's face. The hands-to-face detector 32 can also comprise a plurality of infrared sensors positioned within a bracelet pointing towards the user's wrist and configured to detect a hands-to-face movement based on an image of the wrist captured by said infrared sensors.

The controller 14 is configured to receive data obtained by the hands-to-face detector 32 and to process this data to determine whether there is an attempt by the user (or another person) to reach out to the user's face using hands. If it is the case, then the controller 14 sends a signal to the face shield unit 16 for generating a notification signal (alarm signal). The detection of the hands-to-face gesture by the hands-to-face detector 32, the processing of the information by the controller 14 and the generation of the notification signal by the notification unit 24 are all conducted in real time and very quickly within a very short time period (preferably less than 1-2 second) in order to block the hands before reaching out to the face.

The Health & Safety Monitoring Unit 12 preferably further comprises a Social Distance Detector 34 for detecting presence of an intruding person within a predefined minimum social safety distance threshold of the user. The social distance detector 34 preferably comprises one or more proximity sensors, such as ultra sound sensors, infra-red sensors or laser sensors to measure the distance between the user and the intruding persons. The social distance detector 34 may also use an ultra-wide band technology to measure the distance, particularly when the intruding persons are other users using similar SFPDs 10 such that these SFPDs 10 can communicate with each other to exchange data based on which the speed of the data communication can be measured to determine the distance between these various SFPDs 10.

The notification unit 24 is configured to generate a notification signal (alarm signal) when the intruding person is detected within the predefined minimum social safety distance threshold. The social safety distance threshold is normally in the range of 6 feet, however it is preferable to be set in the range of 6-10 feet to be more on the safe side. The predefined minimum social safety distance is set based on the recommendations of the world health organization for the prevention of the COVID-19 and other viruses of similar nature. The detection of an intruding person by the social distance detector 34, the processing of the information by the controller 14 and the automatic adjustment of the face shield unit 16 into a secured position are all conducted in real time and very quickly within a very short time period (preferably less than 1-2 seconds) in order put the face shield unit 16 in the secured position before the intruding person gets to a critical infectious position.

The Health & Safety Monitoring Unit 12 preferably further comprises a Face Shield Use Detector 36 for detecting and providing an indication on whether the user is wearing the SFPD 10 which is particularly useful in case the SFPD 10 is head wearable by the user (such as a child) and the wearing of the SFPD 10 needs to be monitored by a remote administrator. The Face Shield Use Detector 36 can comprise any suitable sensor, such as a proximity sensor or an infra-red sensor which can detect whether the user is wearing the helmet with the SFPD 10. The infra-red sensor can be the same infra-red sensor used to measure the body temperature of the user. In fact, if the infrared sensor reading is below a human body temperature (for example 37 degrees and below), this is indicative of the absence of human body which can be interpreted as a non-use of the SFPD 10 by the user. Other suitable sensors can be used such as a switch button which is activated when the SFPD 10 is worn and deactivated otherwise. The notification unit 24 directly (or through the controller 14) receives an indication from the Face Shield Use Detector 36 on whether the face shield is being used by the user. The controller 14 can store the information in a memory 18 and/or can notify through the user interface 20 and/or send a signal to a remote administrator informing of the same depending on the application and configuration of the SFPD 10. For example, in the case of school children required to wear head wearable SFPDs 10, a school administration can monitor whether children are wearing their head wearable SFPDs 10, and when a given child is not wearing their SFPD 10, the Face Shield use Detector 36 detects that the SFPD 10 is not being worn by the child and this information is fed to the controller 14 which sends a notification signal (alarm signal) which appears on the SFPD's user interface 20, and preferably a notification is sent to an admin control device 80 (held by the school administration) through the notification unit 24.

The Health & Safety Monitoring Unit 12 preferably further comprises a positioning/crowd detector 38 for tracking and recording the location of the user. The positioning/crowd detector 38 preferably comprises a global positioning system (GPS) device or another positioning technology to track and locate the geographical location of the user, including in an indoor environment. In an exemplary embodiment of the invention, SFPDs 10 are provided to one or more groups of users within a community controlled by a community administration (for example children at a school controlled by a school administration or patients at a hospital controlled by a hospital administration) such that each user is associated to a respective SFPD 10. The locations of the users are tracked through the positioning/crowd detector 38 of their respective SFPDs 10 which obtains the positions of the users in real time and sends it to a crowd detection module within an SFPD administration system 40 which maps the real time positions of the users and generate a crowd map showing the crowd densities with a highlight on any overcrowded area to be avoided by other users or to be inspected by the community administration.

In an embodiment of the invention, the face shield unit 16 comprises a face shield 168 which comprises a moveable face shield portion configured to be automatically adjusted between a secured and unsecured positions (close and open the face shield respectively, for example) based on a command signal received by the SFPD 10.

In an embodiment of the invention, the automatic adjustment is conducted without a manual force by a user, preferably through a motor 164 connected to the face shield 168 for automatically opening and closing the face shield 168 based on the command signal. The command signal can be originate from the user, or can be generated automatically by the SFPD 10 at the occurrence of a certain event such as the detection of a H&S condition associated to the user or a person passing in proximity of the user.

In an embodiment of the invention, the face shield 168 has a face shield opening and where the moveable portion of the face shield 168 is configured to cover the face shield opening when the SFPD 10 is in the secured position, and to uncover it otherwise when the SFPD 10 is in the unsecured position.

Preferably, in an embodiment of the invention, the command signal is a command signal from the user, such as for example a voice signal, a touch signal, or a text signal.

In an embodiment of the invention, the command signal is a voice signal such as “open shield” and “close shield” for respectively opening and closing the moveable part of the face shield, or any other commands as suitable.

In an embodiment of the invention, the command signal received by the SFPD 10 is based on the health and safety conditions of the user or persons passing in proximity of the user.

In an embodiment of the invention, the automatic adjustment (opening and closing the face shield) is conducted without any human intervention based on a command signal received by the SFPD 10 based on the health and safety conditions of the user or persons passing in proximity of the user.

In an embodiment of the invention, the SFPD 10 comprises a safety feature to ensure the safety operation of the opening and closing of the face shield 168, such as for example a motion sensor to detect a motion of the user's hands across the face shield opening and restrict the face shield 168 from closing when/if it is the case.

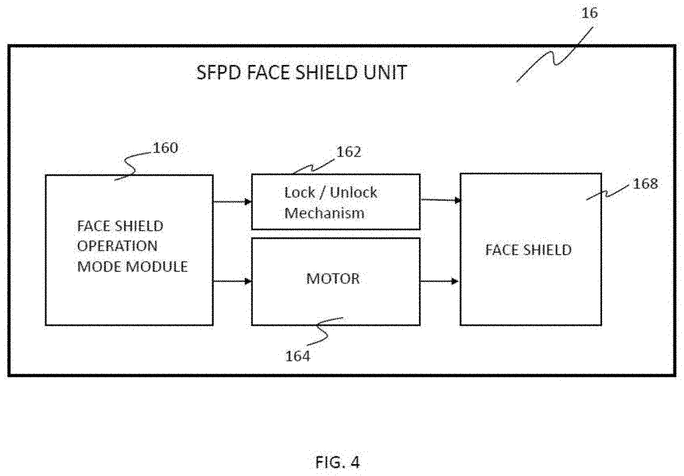

Referring to FIG. 4, in an embodiment of the invention, the SFPD 10 comprises a Face Shield Unit 16 comprising an adjustable face shield 168 (adjustable between a secured/closed position and an unsecured/open position), a face shield operation mode module 160 for setting the operation mode of the face shield among a manual operation (where the face shield 168 is manually operated by the user either to be in the secured position or in the unsecured position based on a manual force or a command signal for example), a secured mode where the face shield 168 is locked in the secure position (meaning the face shield 168 is secured/closed and locked in that position), an unsecured mode where the face shield 168 is locked in the unsecured position (meaning the face shield 168 is unsecured/opened and locked in that position), an automatic mode (where the face shield 168 is automatically operated and adjusted by the controller based on the health and safety condition of the user). The face shield operation mode module 160 obtains the instruction on the operation mode from the controller 14.

The face shield unit 16 further comprises a lock/unlock mechanism 162 and a motor 164 connected to the face shield operation mode module 160 and to the face shield 168. The lock/unlock mechanism comprises a mechanical or an electromagnetic clutch for locking the face shield 168 in a given position (unsecured or secure position) which is also adapted to be deactivated to return the face shield 168 in an unlocked position. The lock/unlock mechanism 162 obtains the lock/unlock instructions from the face shield operation mode module 160. The motor 164 is preferably an electrical motor connected to the face shield 168 for automatically adjusting the face shield 168 between a secure position (for example by closing the face shield) and an unsecured position (for example by opening the face shield). When the face shield operation mode is set to be automatic, the motor 164 automatically adjusts the face shield 168 between a secured and unsecured position depending on the health and safety condition of the user as instructed by the controller 14. When the face shield operation mode is set to be manual, the motor 164 is receptive to a manual instruction by the user or by the administrator to either put the face shield 168 in a secure position (by closing the face shield for example) or to put it in the unsecured position (by opening the face shield for example).

In an exemplary embodiment, SFPDs 10 are used by a group of children at school class where each child is provided by a corresponding SFPD 10 as a personal protective equipment. The teacher (as administrator of the SFPDs 10) can lock the face shields 168 of the SFPDs 10 in the secure position where the all the face shields 168 of the SFPDs associated with the children will be secured/closed and locked in that position in the sense that the children will not be able to unsecured/open the face shields 168 manually. This mode can be set when the children are in a group activity in physical proximity to each other for example. The face shields 168 can also be locked in an unsecured position (all shields open) when the kids are seated on their distant desks during a lecture for example. The teacher can also chose to set the face shields 168 in an automatic mode where the face shields will open and close based the monitoring of the health and safety conditions related to the children. This mode can be set when the children are in a free activity outside the classroom for example.

In an embodiment of the invention, the SFPD 10 comprises a memory 18 is in data communication with the controller 14 for storing and providing reading access to a unique identifier enabling to uniquely identify the user and/or the SFPD 10 associated thereto. The memory 18 is also configured to store information about health & safety conditions associated to the user such as body temperatures of the user, the identity and the body temperatures of persons with whom the user was in direct contact during a specific period of time (also called the intruding persons), the distances between the user and the intruding persons with an indication of any breach of the minimum social safety distance threshold by the intruding persons, the locations of the user during a specific period of time or during a specific incident for example while a high risk intruding person got into direct contact with the user, the crowd density in the various locations of the user during a specific period of time and an indication on whether the user was in a highly crowded area which may be indicative of a high risk, a count of the hands-to-face gestures/block actions by the face shield and any failures to block the hands by the SFPD 10, and so on. The memory 18 can store very valuable health and safety data related to the user (which may be in relation to the user him/herself or to the intruding persons) which may be tracked/accessed at a time in the future to analyze and determine any health and safety vulnerability or breach or to track persons who were in direct physical contact with the user.

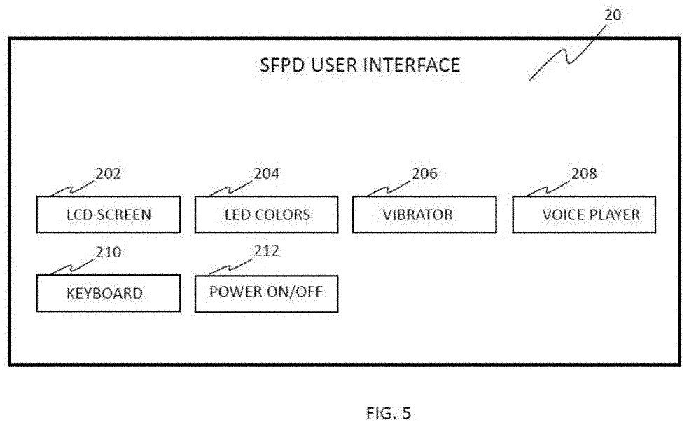

Referring to FIG. 5, the SFPD 10 comprises a user interface 20 (also called user input/output unit) comprising a LCD screen 202, LED colors 204, a vibrator 206, a voice player 208, a keyboard 210 and a power on/of 212. The user interface 20 is in data communication with the notification unit 24 and the controller 12 for inputting/outputting data between the controller 12 and the user interface 20 to/from the user and surrounding persons. For example, the controller 12 can activate the vibrator 206 to alert the user when an intruding person is approaching in proximity of the user in breach of the social safety distance. Based on the alert, the user can stop, change direction or take any other precautionary measure. The controller 12 can also activate the voice player 208 to play a voice message to the user or to the intruding person alerting about the breach. The voice message can also be customized based on the identity of the intruding person. For example, in case where the SFPDs 10 are used by school kids, a first SFPD 10 associated to a first kid obtains the identity of an intruding kid by way of data communication between the first SFPD 10 (associated to the first kid) and a second SFPD 10 associated to the intruding kid. The identity of the intruding kid is stored inside the memory 18 of the second SFPD 10 and transmitted to the first SFPD 10 through the inter-device communication unit 22. In this case, the voice message played by the first SFPD 10 can be customized to mention the name of the intruding kid, such as “Maria, please keep distance”. The voice message when customized to mention the name of the intruding person will be perceived as friendlier and less intrusive particularly for kids. The LCD screen 202 can be used to display a message which can be updated on a real time basis. The LED colors 204 can be used to identify groups of users. For example, school kids can be divided into various groups where each group is given a specific color code which is displayed through the LED Colors 204. This is as social distancing is becoming more and more important at schools, should administration might need to divide kids into different groups each of which have a different time schedule for using public areas such as the playgrounds, the library and the bathrooms. If a group of kids identified by a “blue” color for example are scheduled to play at the playground at a specific time period, it will be easy to identify through the color code any breach of the schedule by other kids not belonging to the same group. If a kid with “red” color appears in the playground, this kid will be easily spotted as breaching the social distancing/scheduling rule. The keyboard 210 can be used to set/change some configurations of the SFPD 10 such as the user identity, the temperature threshold, the minimum social safety distance threshold, the color codes, and so on. The power on/off 212 will enable to switch on and off the SFPD 10.

The SFPD 10 comprises a notification unit 24 in electrical communication with the H&S Monitoring Unit 12 (directly or through the controller 14) configured to notify a health/safety risk condition such as a high temperature above the predefined temperature threshold or a breach of the minimum social safety distance. The notification unit 24 is configured to notify the user or other users through a user interface 20 such as a screen 202, a led 204, a voice message 208, or a vibrator 206. In an embodiment of the invention, the notification unit is configured to be connected to a remote administration control device 80 through a data network (such as WiFi) to notify an administrator (such as a teacher or school director) of presence of a health/safety risk associated to the user.

The SFPD 10 comprises s communication unit 22 (also called inter-device communication unit) in electrical communication with the controller 14 and with external remote devices through a data network. In an embodiment of the invention, the inter-device communication units 22 associated to the various face protective devices 10 are configured to exchange health/safety data relative to their respective users (for example the identity of the intruding persons passing within the social safety distance threshold, and the temperature of the intruding users) and to store this data inside the memory 18 of their respective SFPDs 10.

In an embodiment of the invention, the data exchanged by a SFPD 10 comprises an indication of any health/safety risk associated to its respective user (for example an indication of high temperature of the user, or a measure of the temperature itself), and wherein the SFPD 10 automatically adjusts the face shield unit 16 in the secured position (for example by closing the face shield 168) when/if the temperature of the intruding person as obtained by the communication unit 22 is above the predefined temperature threshold. Preferably, the identity of the intruding user (or associated SFPD 10) is stored inside the memory 18 of the user's SFPD 10 which can be retrieved at any future time for tracking and identification purposes of infected persons.

The Health & Safety Monitoring Unit 12 can comprise an external component (system or device) located remote from the SFPD 10. In this case, the communication between the controller 14 and the external component/device of the Health & Safety Monitoring Unit 12 is a remote wireless communication (through a data network such as WiFi or Bluetooth or any other suitable wireless communication technology) conducted through the communication unit 22 of the SFPD 10.

In an exemplary embodiment, the external component of the Health & Safety Monitoring Unit 12 comprises an identification system configured to identify the user. The identification system can for example be an RFID system wherein the RFID reader can be located at the remote (external) component of the Health & Safety Monitoring Unit 12 and the RFID tag is located at the SFPD 10. The external component of the Health & Safety Monitoring Unit 12 identifies the user, monitors his body temperature and sends a signal to the SFPD 10 of the user (through the controller) in order to automatically adjust the face shield 168 in the secured position when the temperature of the user is beyond the predefined temperature threshold. In another embodiment, the external component comprises the thermal detector 30 of the Health & Safety Monitoring Unit 12 which screens the body temperature of other persons passing or being located in proximity of the user (the intruding persons), and sends a signal to the SFPD 10 of the user (through the communication unit 22 connected to the controller 14) in order to automatically adjust the face shield 168 in the secured position when the temperature of an intruding person is beyond the predefined temperature threshold.

The SFPD 10 comprises an admin control unit 26 in electrical communication with the controller 14 and with a remote admin control device 80 through a data network. The admin control unit 26 is adapted to receive remote instructions from the remote admin control device 80 with respect to the operation and control of the SFPD 10. In an embodiment, the admin control unit 26 receives an activation and deactivation signals to respectively activate and deactivate the SFPD 10. The admin control unit 26 is adapted to activate/deactivate the SFPD 10 based on the received signal from the admin control device 80. In another embodiment, the admin control unit 26 receives a notification or a message from the administrator which can be communicated to the user or surroundings via the user interface 20. The message or notification is processed by the controller 14 and then communicated to the user interface 20. In another embodiment, the admin control unit 26 receives instructions from the admin control device 80 with respect to the operation mode of the face shield unit 16 (for example, manual, automatic, locked secured or locked unsecured). These instructions are sent to the controller 14 for processing, where the controller 14 controls the SFPD face shield unit 16 based on these instructions. In another embodiment, the admin control unit 26 comprises a pairing module adapted to pair the SFPD 10 with a given admin control device 80. In one example, the pairing is conducting through frequency alignment/pairing between SFPD 10 and admin control device 80. In another embodiment, the admin control unit 26 receives a labelling signal to label the SFPD 10 for example through the allocation of a given color code to the LED colors 24 of the user interface 20. This is to be able to identify users as part of a given group (labelled by the given color code) among various groups.

The SFPD 10 comprises a power source 28 which is preferably a rechargeable battery and adapted to supply power to all the electrical components of the SFPD 10.

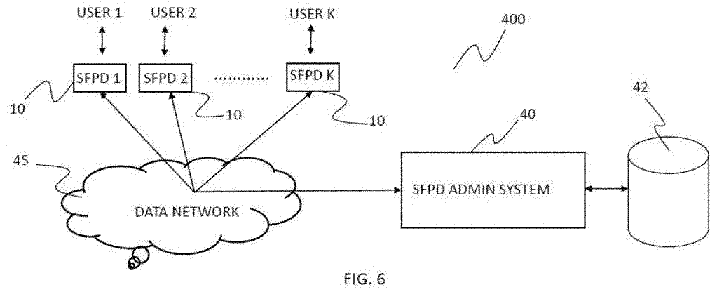

As another aspect of the invention, referring to FIG. 6, there is provided a SFPD network 400 comprising a plurality of smart face protective devices (SFPDs) 10 associated to respective users within a given community (such as a school or a hospital), a SFPD Admin System 40 connected to the SPFDs 10 through a data network 45, and a SFPD Admin Database 42 connected to the SFPD Admin System 40 for storing data related to health and safety conditions of the users as received from the SFPDs 10 which enables the tracking and analysis of health and safety records in connection with the users. The SFPD Admin System 40 is configured to obtain the data stored within the memory 18 of the SFPDs 10, to compile and analyze the obtained data based on a predefined health data management algorithm and to output precautionary/proactive health measures and/or corrective/reactive health measures in connection with the users. For example, if a particular person is diagnosed COVID-19 positive (infected person) in a particular period, it will be possible through the stored records in the database 42 and the SFPD admin system 40 to identify the other persons at risk and take measures to reach out and test these persons at risk. This can be conducted by for example tracking back all others persons within the community who have been into direct physical contact with this infected person.

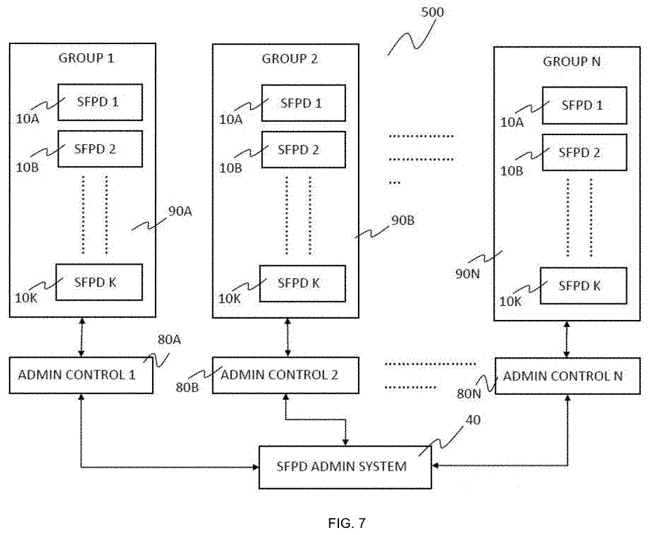

As a further aspect of the invention, referring to FIG. 7, there is provided a SFPD Network 500 comprising:
N number of SFPD Groups (90A, 90B, . . . , 90N), where each SFPD Group (90A, 90B, . . . , 90N) comprises K number of SFPDs (10A, 10B, . . . , 10K);
N number of SFPD Control Devices (80A, 80B, . . . , 80N), where each SFPD Control Device (80A, 80B, . . . , 80N) is connected/configured to be connected to one SFPD Group (90A, 90B, . . . , 90N);
a SFPD Admin System 40 connected/configured to be connected to the N number of SFPD Control Devices.
where N≥1 and K≥1; however, preferably N≥2 and K≥2; and more preferably N≥3 and 3≥K≥5 (for minimizing grouping between individuals and increasing social distancing);

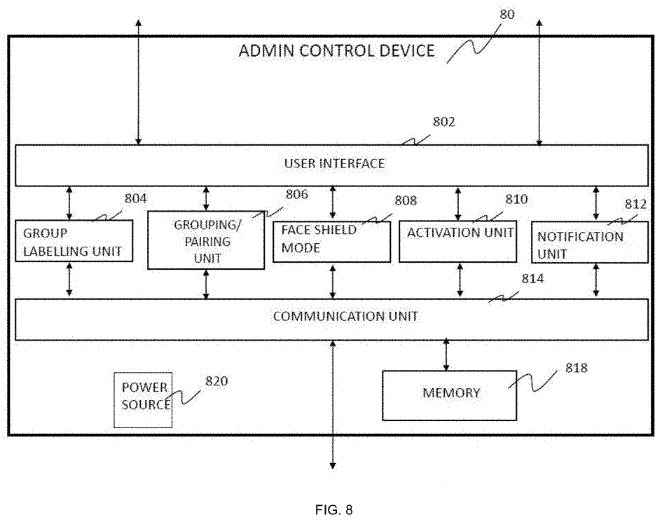

Referring to FIG. 8, the Admin Control Device 80 comprises a user interface 802, a group labelling unit 804, a grouping/pairing unit 806, a face shield mode 808, an activation unit 810, a notification unit 812 and a communication unit 814.

The user interface 802 is connected to the grouping/pairing unit 806 for enabling the identification/selection of the K SFPDs (10A, 10B, . . . 10K) to be paired/connected to the Admin Control Device 80. This identification/selection can be conducted by entering the unique identifiers associated to the K SFPDs (10A, 10B, . . . 10K) or through a pairing technique between the K SFPDs (10A, 10B, . . . 10K) and the Admin Control Device 80. The user interface 802 and the grouping/pairing unit 806 are configured to enable the selection/identification of the K SFPDs (10A, 10B, . . . 10K) which are to be paired to that specific Admin Control Device 80.

This process is repeated for each one of the N SFPD Groups (90A, 90B, . . . , 90N) for enabling the pairing between each one of the N Admin Control Devices (80A, 80B, . . . , 80N) associated to a specific group among the N SFPD Groups and its respective K SFPDs (10A, 10B, . . . , 10K) such that each Admin Control Device among the Admin Control Devices (80A, 80B, . . . , 80N) is paired to K SFPDs (10A, 10B, . . . 10K) and this is repeatedly for each and every one of the N SFPD Groups.

For example, a school of 100 children can be divided in 20 groups, each group comprising 5 kids. The kids of the school are provided with SFPDs 10 such that each kid has a corresponding SFPD 10 uniquely identified under his name. The total number of SFPDs 10 is equal to the total number of kids which is 100. The total number of SFPD Groups 90 formed in this case is the total number of groups formed with the kids which is N=20. The total number of Admin Control Devices 80 in this case is N=20. The total number of SFPDs 10 selected/identified under each group in this case is K=5. In this example, there is provided 20 Admin Control Devices 80 where each Admin Control Device 80 is configured to be connected/paired to 5 SFPDs 10 respectively for each one of the 20 SFPD Groups formed for the 20 Groups of Kids. Each Admin Control Device (80A, 80B, . . . ,81K) enables the identification/selection/pairing of the K SFPDs associated to a given SFPD Group among the N Groups, such that the K SFPDs in that given group can communicate exclusively with that specific Admin Control device 80 to which they have been paired. Any message or notification transmitted from a SFPD 10 will be received by the corresponding Admin Control Device 80 to which it has been paired.

The grouping/pairing enables one Admin Control Device 80 (for example Admin Control Device 80B) to simultaneously operate and control the K SFPDs paired with that specific Admin Control Device 80B. For example, the administrator of the Admin Control Device 80B can provide the green labelling code to his group of kids using the Admin Control Device 80B which will automatically be communicated to/and reflected on the SFPDs paired to that specific Admin Control Device 80B. This simultaneous control/management of the SFPDs 10 is applied to all other functions and features provided by the Admin Control Device 80 for the management/control/administration of the SFPD 10 which includes the group labelling provided by the group labelling unit 804, the face shield operation mode which is provided by the face shield mode module 808, the activation/deactivation of the SFPD 10 which is provided under the activation unit 810, the notification of the users through alert communication/notifications which is provided under notification unit 812.

The user interface 802 is connected to the group labelling unit 804 for enabling the unique labelling of the SFPD 10 according to a specific labelling code or name. For example, this can be done through a color code which is communicated to the SFPDs 10 and displayed by the LED Colors 204 of the SFPD 10. In the example set out above, the 10 groups can be given 10 different color codes where each SFPD 10 among the 100 SFPDs 10 would display by its respective LED Colors 204 the color related to the group to which it belongs. In this example, the SFPDs 10 in a particular SFPD Group 1, 2, . . . , N will have a color code 1, 2, . . . N respectively. The labelling can also be done through name appellation, such as “lion class” or “tiger class” or “elephant class” which is displayed on the LCD screen 202 of the SFPD user interface 20.

The user interface 802 is connected to the face shield mode module 808 to enable the administrator to select the operation mode of the face shield unit 16 (manual, automatic, locked secured or locked unsecured). This mode is communicated to the SFPDs 10 through the communication unit 814 and used to operate the SFPD Face Shield Units 16.

The user interface 802 is connected to the activation/deactivation unit 810 to enable the administrator to activate/deactivate (turn on/turn off) the SFPDs. This mode is communicated to the SFPDs 10 through the communication unit 814.

The user interface 802 is connected to the notification unit 812 for receiving any message or alert from the SFPD 10 (such as an alert of high temperature). This notification is communicated to the administrator through the user interface 802.

The Admin Control Device 80 is configured to receive the health and safety user related data stored inside the memory 18 of the corresponding SFPD 10 to which it is paired to store this information inside the memory 818 of the Admin control Device 80.

The Admin Control Device 80 also comprises a power source 820, preferably a rechargeable battery, for providing power supply to the various electronic components of the device 80

The Admin Control Device 80 is configured to communicate with the SFPD 10 through the Admin Control Unit 26 which is configured to receive the various signals from the Admin Control Device 80 and convert them into signals comprehensive by the controller 14 of the SFPD 10.

The N Admin Control Devices (80A, 80B, . . . , 80N) are connected/configured to be connected to the SFPD Admin System 40 through a data network (preferably wireless) for transmitting the health and safety user related data to the SFPD Admin System 40 for storage at the database 42 and further processing and analysis.

The SFPD Admin System 40 is also configured to control/manage the N Admin Control Devices (80A, 80B, . . . , 80N) for controlling the SFPDs 10. According to this embodiment, all the instructions received through the user interface 802 for the operation of the various modules and units 804, 806, 808, 810 and 812 are rather received from the SFPD Admin System 40 through the communication unit 14.

The present disclosure also provides a smart face shield 100. A user may wear the smart face shield 100 to prevent or reduce the spread of infectious diseases. The infectious diseases (interchangeably used as “virus”) may include but is not limited to the novel COVID-19 coronavirus.

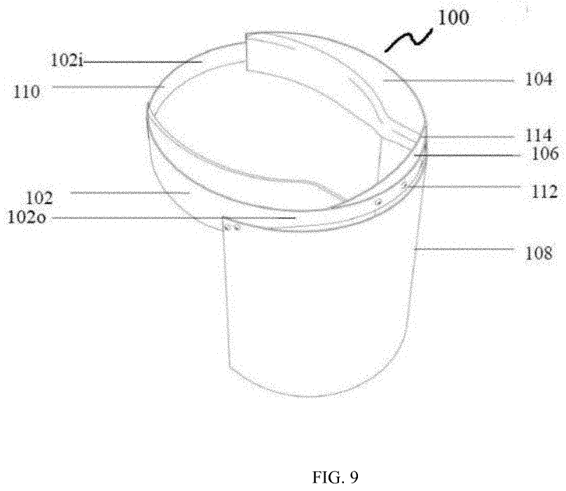

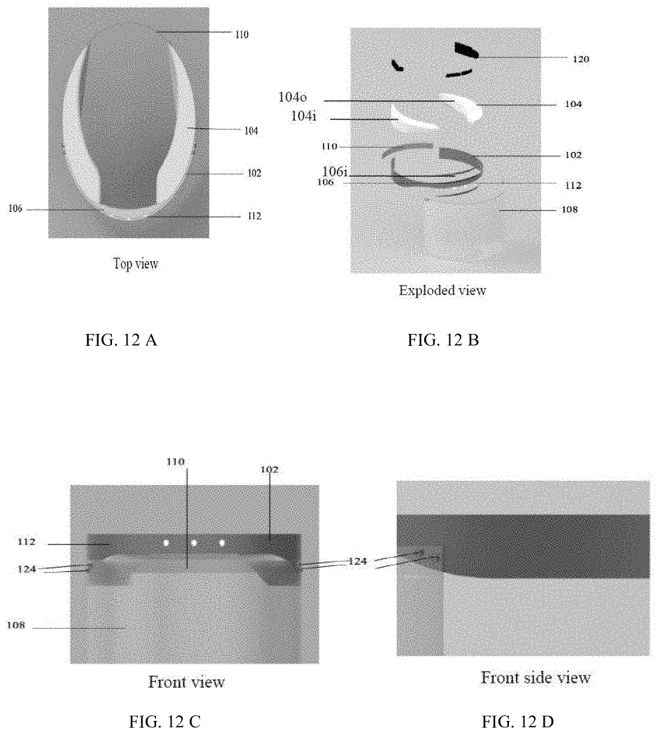

FIG. 9 illustrates an overall schematic view of a smart face shield 100, in accordance with an embodiment of the present invention. The device 100 may comprise a shell 102, a pad 104 configured to be fixed to the shell 102 and a circuitry 106 to operate the smart features of the smart face shield 100. The pad 104 may allow housing of the circuitry 106. FIG. 12 illustrates different views of the smart face shield 100, in accordance with an embodiment of the present disclosure.

In an embodiment, the shell 102 may be made of a sheet, preferably a bendable material to form a U-shaped shell 102. The shell 102 may form a shell kind structure of U shape or semi circular shape. The bendable material may include but is not limited to bendable plastic material, aluminum, stainless steel. The U shape or semi circular shell 102 can also be made of any other suitable material, such as polymer, having flexible/bendable properties resistant to breaking such that the U shape shell 102 may be adjusted (thanks to the bendable/flexible properties) when being worn to various sizes of user heads without being broken. Preferably, the shell 102 is light in weight.

According to an embodiment, the shell 102 may be bent and laser cut shell from 0.6-1.0 mm sheet. The shell 102 may have an inner surface 102i and an outer surface 102o. The pad 104 may further comprise an inner surface 104i and an outer surface 104o such that the outer surface 104o of the pad may be fixed to the shell 102, particularly, the inner surface 102i of the shell, with hook-and-loop fastener (not shown in the figure) and the inner surface 104i of the pad may come in contact with the head of the user 122. The pad 104 may be made of foam or other soft material according to an embodiment. The foam may be lining foam or memory foam.

The pad 104 may be U shaped or semi-circular shaped with the radius being less than the radius of the shell 102. In another embodiment, the pad 104 may comprise two curved portions connected with a hook-and-loop fastener at each of the two ends of the curved portions.

In an embodiment, the pad 104 may comprise a cover, and preferably a garment cover that may have a socket adapted to receive the shell 102 such that the pad 104 is removably connected to the shell 102. The garment cover further comprises a pocket to receive the pad. The garment cover may be made of a washable material according to an embodiment. This would keep the pad clean while the device is being worn by the user since the pad would not be in direct physical touch contact with the head of the user. This would avoid sweat and dirt and keep the pad clean and hygienic. This is of particular importance when the pad is made of material unsuitable to be washed on a daily basis. The garment cover may be removed from the shell and the pad when desired for clearing or washing purposes for example, making the device highly hygienic for daily use. In an embodiment, the process of mounting the pad to the device when desired comprises inserting the pad inside the pocket of the garment cover and inserting the shell inside the socket.

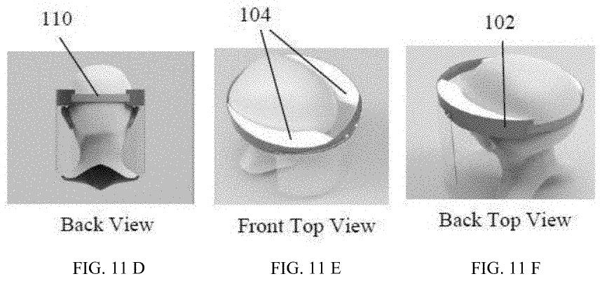

FIGS. 11 A-F illustrate different views of the device 100 worn by the user 122 in accordance with an embodiment of the present disclosure, the pad 104 may comprise a forehead housing 114 positioned at about the forehead of a user 122 when the device 100 is being worn. The pad 104 is configured 104 to keep the forehead housing 114 at a predefined distance from the forehead of the user 122 while the device 100 is being worn, to allow contactless distance between the forehead housing 114 and the circuitry 106. The forehead housing 114 may be configured to be received at a first part 106i of the circuitry 106. The first part 106i of the circuitry 106 may correspond to the inner surface of the circuitry 106. The circuitry 106 may comprise one or more sensors for real-time sensing of several parameters.

In an embodiment, the first part 106i of the circuitry 106 may comprise a contactless heat sensor configured to detect the user's temperature using the user's forehead. Therefore, the device 100 may offer advantage of determining the temperature of the user 122 indicating his/her health condition without the requirement of any other person coming in contact of the user 122. The first part 106i of the circuitry 106 may further comprise a contactless heat sensor. Furthermore, the first part 106i of the circuitry 106 may also comprise a proximity sensor for detecting a proximity distance with an intruding person in proximity of the user. The proximity sensor may be a laser sensor configured to measure the distance of the user from the intruding person. The proximity distance may be a predefined and pre-set distance.

Further, the device 100 may comprise an indicator panel 112 in the circuitry 106. The indicator panel 112 may comprise a plurality of indicators 112a positioned on the outer surface 102o of the shell 102 and may be above the forehead housing 114. Each of the indicators 112a may be in connection with the sensors in the circuitry 106 and each of the indicators 112a may provide a signal in response to the observations of the sensors. The signal may be provided by a blinking LED, or a LED constantly glowing, or may be a combination of LED with a siren blown up. For example, an indicator connected to the temperature sensor may provide a signal when the temperature of the user 122 is beyond the predefined temperature. In another example, an indicator 112a connected to the proximity sensor may provide a signal when the intruding person is at a distance less than the predefined threshold distance from the user 122. In an embodiment, the predefined temperature threshold may be around 38.5 degrees Celsius and the predefined social distance threshold may be around 2 meters. The signal may be a light, a sound, a vibration or a wireless signal sent to a remote device for example.

Further, the device 100 may comprise a removable shield 108 (interchangeably called as shield) positioned at front and connected to the shell 102. The shield 108 may be made of a transparent material including but not limited to polyethylene terephthalate glycol (PETG). The shield 108 made of PETG may also be painted, silk screened or hot stamped. The shield 108 may be cut in a rectangular shape and curved through the portion with shorter length so as to make an arc shaped structure. The curved portion may then reattach to the outer surface of the forehead housing 114. The connection may be made at the two ends of the shield 108 through, for example, hook-and-loop fasteners 124 or any other suitable coupling mechanism such as screws. The shield may be removable attached to the shell 102 such that it may be detached and replaced when damaged or when to be cleaned.

Furthermore, the device 100 may comprise an adjusting unit 110 positioned at the back side of the device 100. The back side may be opposite to the side where the indication panel 112 may be positioned. The adjusting unit 110 may comprise an adjustable size fabric fastener, the two ends of which may be connected to the respective ends of the shell 102. In an embodiment, the fastener may be made of an elastic material such that it adjusts itself according to the head of the user when worn and stays stable while the user moves. In another embodiment, the fastener may have an adjusting mechanism comprising multiple predefined adjusting positions on the length of the fastener. The user may opt for any of the predefined positions based on his/her comfort while wearing the device 100. In an embodiment of the invention, the adjusting unit has a hollow inner space for allowing the passage of a wire for connecting circuitry components distributed at opposite sides of the device with respect to the adjusting unit 110.

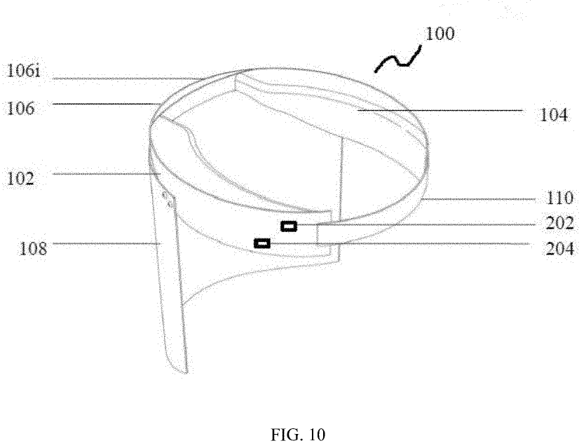

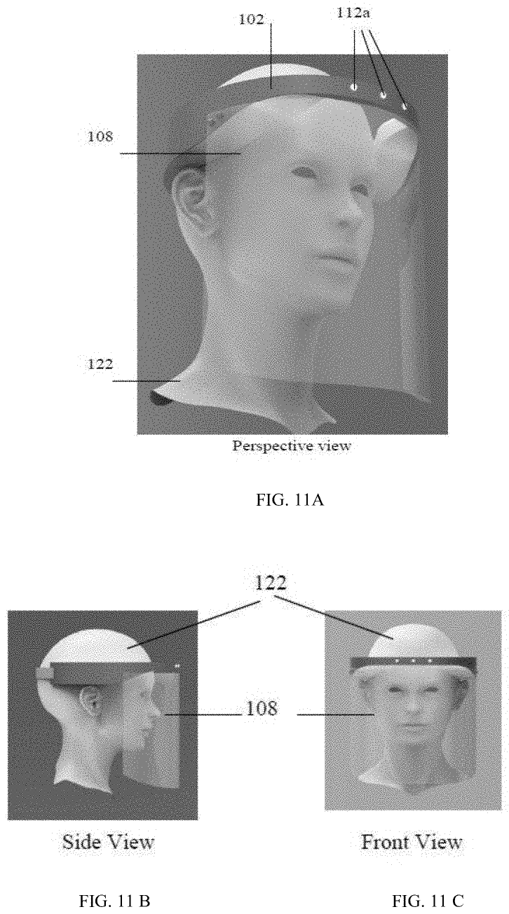

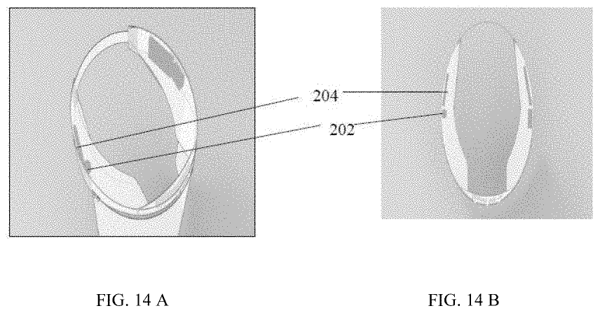

FIG. 10 illustrates a back view of the smart face shield 100, in accordance with an embodiment of the present disclosure. The smart face shield 100 comprises a switching button 202 at the outer surface 102o of the shell unit 102. For ease of the user 122, the switching button 202 may be positioned near one of the ends of the adjusting unit 110. The user may switch ON/OFF the circuitry 106 by operating the switching button 202. The circuitry 106 may be put to OFF position when not in use or when the user 122 intends not to use the sensors. This may enable prevention of wastage of energy by the device 100. FIGS. 14A and 14B illustrates different views of the positioning of circuit elements in the smart face shield, according to an embodiment of the present disclosure. In an embodiment of the invention, the circuit components may be positioned within the pad 104. The pad 104 may have openings to enclose/house the components at various positions as suitable depending on the circuitry component. Some of the openings may be made through the inner surface 104i of the pad such that electronic components enclosed therein may be accessible through the shell 102. This may be the case for the switching button 202 and the USB port 204. Some openings may be made within the internal portion of the pad (the portion between the inner periphery 104i and the external periphery 104o of the pad 104). In this case, the pad 104 provides an isolation (including heat isolation) to the electronic components enclosed within the openings formed within the internal portion of the pad 104. These openings (within the internal portion of the pad 104) may be formed by cutting the pad 104 along a dissection line, making openings within the surface of at least one of the dissected portions.

In an alternative embodiment, the pad 104 may also be made of two distinct portions comprising openings suitable to house/enclose the electronic components which are then coupled together once the electronic components are placed therein. The coupling may comprise gluing both portions together or using a zipper for bringing both portions together or using any other suitable coupling mechanism. In an embodiment, the pocket of the garment cover configured to receive the pad 104 may comprise two separate pockets, a first pocket portion for receiving the first portion of the pad and second pocket portion for receiving the second portion of the pad. The pocket shall be made of suitable material and dimensions for brining both portions of the pad 104 together in a symmetric manner without offset such that one pad portion doesn't slide away from the second pad portion.

In an embodiment, the pad 104 may have perforations to enable the dissipation of heat originating from the electronic components outside the device.

Further, the device 100 may comprise a USB port 204 positioned on or accessible through the shell unit 102. The USB port 204 may allow charging of the device 100 on being connected to a host device. The host device may be a personal computer according to an embodiment of the invention. Additionally, the USB port 204 may enable the user 122 to upload data to and/or download data from the host device. The user 122 may be able to personalize the device 100 by himself setting the preset values for the sensors. This may enhance the user experience associated with the device 100.

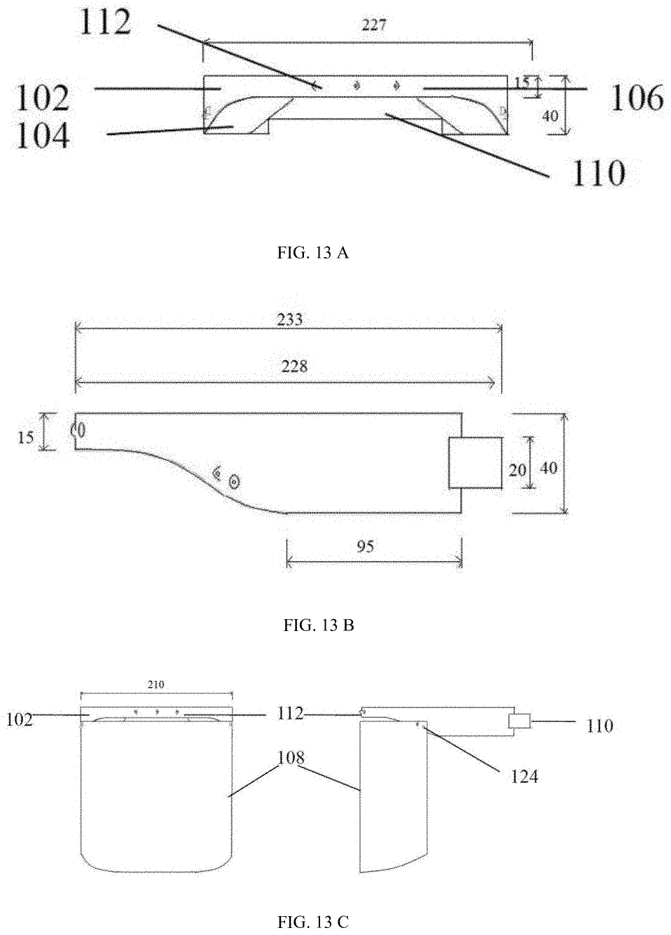

FIGS. 13 A-C illustrates dimensions corresponding to the smart face shield 100, in accordance with an embodiment of the present disclosure. The width of the shell 102 may be 40 mm at the sides and 15 mm at front. The pad 104 as connected to the shell 102 at the sides may be 40 mm in width. Referring to the side view of the device 100 as illustrated by FIG. 12, the total length of the side portion measured from the middle of the indicator panel 112 at the front of the shell 102 to the middle of the adjusting unit 110 at the back side of the shell 102 may measure 233 mm in length while the length of the side portion measured from the middle of the indicator panel 112 at the front of the shell 102 to the back side of the shell 102 and till the middle of the pad 104 (excluding the adjusting unit 110) may measure 228 mm. According to an embodiment, out of 233 mm, the length of the side as measured from the middle of the adjusting unit 110 at the backside of the device 100 to the location where the shield 108 is attached to the shell may measure 95 mm. Further, the fabric fastener of the adjusting unit 110 may be 20 mm wide according to an embodiment. Further, the front length of the circular shell 102 that may be positioned on the forehead of the user may measure 227 mm and the straight portion of the front length of the circular shell 102 may measure 210 mm. In other words, the straight length of the front view of the smart face shield 100 may measure 210 mm at the front of the user's forehead and the total front length of the shell 102 may measure 227 mm.

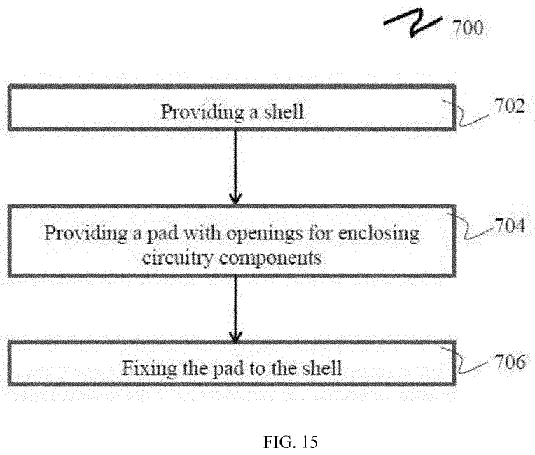

FIG. 15 illustrates a flow diagram depicting a process 700 of manufacturing a face shield 100, in accordance with an embodiment of the present disclosure. At step 702, the process 700 may comprise providing a shell 102 in a U shaped configuration such that the shell 102 may be bendable and flexibly mountable around the head of the user 122. Further, at step 704, the process 700 may comprise providing a pad 104. The pad 104 may be cut so as to enable it to receive the electronic components at the inner surface 104i of the pad. At step 706, the pad 104 may be fixed to the shell 102 at the inner surface 102i. Referring to FIGS. 12 (a)-(d), the pad 104 may be fixed to the shell 102 with hook-and-loop fastener (not shown in the figure). According to an embodiment of the present invention, the circuit components may include one or more of arduino nano, Lithium battery charger (Micro USB), resistors, buzzers, tracker, VL53L0X sensor, Slide switch, female to female jumper wires, and MLX90614 contactless temperature sensor.

In an embodiment, the smart face shield 100 may prevent the virus (or other viruses or germs) from the surrounding from contacting the user 122 and also may prevent the virus from spreading from the user 122 to the surrounding.

In an embodiment, the smart face shield 100 is light weight and is comfortable to wear for prolonged hours and also easy to remove off the head of the user 122. The smart face shield 100 is also appealing to the eyes of the user 122, and is particularly attractive to children, which prompts them to wear it and develops safety habits in them. The smart face shield 100 is preferably less than 200 grams, and more preferably less than 150 grams, and even more preferably less than 100 grams.

In an embodiment, the smart face shield 100 is enabled with adjustment in size based on the requirement (head) of the user 122 and is stable even in the circumstances of sudden movement of the user 122. The smart face shield 100 offers easy dismantling of the parts comprised therein, thereby allowing replacing the worn out parts with the new one without replacing the whole face shield. The worn out parts may include but not limited to the shield 108. In an embodiment, the face shield 100 is, therefore, economic, easy to maintain, and has a longer life.

Many changes, modifications, variations and other uses and applications of the subject invention will become apparent to those skilled in the art after considering this specification and the accompanying drawings, which disclose the preferred embodiments thereof. All such changes, modifications, variations and other uses and applications, which do not depart from the spirit and scope of the invention, are deemed to be covered by the invention, which is to be limited only by the claims which follow.

1. A smart face protective device (SFPD) for infection control, wherein the smart face protective device comprises of:
a health and safety monitoring unit, wherein the health and safety monitoring unit comprises of a thermal detector that can measure the temperature of a user and/or a person passing in proximity of the user, a hands-to-face detector that can detect the motion of a hand to the user's face, a social distance detector that can measure the distance between the user and the person in proximity to the user, a face shield use detector that can detect whether the face shield is worn by the user, and a position/crowd detector to track the location of the user.
a Notification Unit, wherein the notification unit generates a notification signal (alarm signal) to a user and/or other users based on a detection of an occurrence of a H&S condition; and
a face shield unit, wherein the face shield unit comprises of a face shield operation mode module, a lock/unlock mechanism, a motor, and a face shield.

2. The smart face protective device of claim 1, wherein the H&S conditions comprise of:
a temperature of the user or a person passing in proximity of the user that is higher than a predefined temperature threshold;
a social distance between the user and another person (intruding person) that is lower than a predefined social distance threshold, and
a motion by the user or another person to touch the user's face.

3. The smart face protective device of claim 1, further comprising of a light, a vibrator, a display, and a sound system.

4. The smart face protective device of claim 3, wherein the smart face protective device produces a light or a sound upon a detection of an occurrence of the H&S condition.

5. The smart face protective device of claim 2, wherein the notification unit is connected a remote administration device through a data network to notify an administrator of a presence of a health/safety risk associated to the user.

6. The smart face protective device of claim 2, wherein the smart face protective device stores data related to the H&S conditions inside a memory.

7. The smart face protective device of claim 1, further comprising a moveable face shield portion configured to be automatically adjusted between a secured position and an unsecured position based on a command signal received by the smart face protective device.

8. The smart face protective device of claim 7, wherein the automatic adjustment is conducted through the motor connected to the face shield for automatically opening and closing the face shield based on the command signal received by the smart face protective device.

9. The smart face protective device of claim 7, wherein the command signal is a voice, text, or touch signal by the user.

10. The smart face protective device of claim 7, wherein the command signal is generated automatically upon a detection of an occurrence of the H&S condition.

11. A smart face shield comprising:
a shell;
a pad; and
a circuitry, wherein the pad is configured to house the circuitry.

12. The smart face shield of claim 11, wherein the pad further comprises a garment cover for covering the pad, wherein the garment cover has a socket adapted to receive the shell for removably fixing the pad to the shell.

13. The smart face shield of claim 11 further comprising a shield connected to the shell.

14. The smart face shield of claim 11 further comprising a forehead housing positioned at about the forehead of a user when the device is being worn, wherein the forehead housing is configured to receive a part of the circuitry,

15. The smart face shield of claim 14, wherein the first part of the circuitry comprises a contactless heat sensor configured to detect the user' s temperature using the user's forehead.

16. The smart face shield of claim 15, wherein the first part of the circuitry further comprises a proximity sensor for detecting a proximity distance with an intruding person in proximity of the user.

17. The smart face shield of claim 14, wherein the pad is configured to keep the forehead housing at a predefined distance from the forehead of the user while the smart face shield is being worn such that the forehead housing does not touch the user's forehead.

18. The smart face shield of claim 17, wherein the circuitry comprises an indicator panel for providing a signal when the temperature of the user is beyond a predefined temperature threshold and when an intruding person is below a predefined social distance threshold from the user.

19. A process of manufacturing a smart face shield, comprising a shell, a pad, and a circuitry, comprising the steps of:
a) providing a shell in a U-shaped configuration such that the shell is bendable and flexibly mountable around the head of a user;
b) cutting the pad for receiving at least one circuit component within the pad; and
c) fixing the pad to the shell.

20. The process of manufacturing a smart face shield of claim 19, wherein the process further comprises the step of including one or more of arduino nano, lithium battery charger (micro US), resistors, buzzers, tracker, VL53L0X sensor, slide switch, and female to female jumper wires, and MLX90614 contactless temperature sensor as circuit components within the pad.
